(19) United States
(12) Patent Application Publication
(10) Pub. No.: US 2026/0021153 A1
(43) Pub. Date: Jan. 22, 2026

(54) TRADITIONAL CHINESE MEDICINE FORMULATION FOR PREVENTING AND TREATING CONSTIPATION-PREDOMINANT IRRITABLE BOWEL SYNDROME (IBS-C)

(71) Applicant: Centre for Chinese Herbal Medicine Drug Development Limited, Hong Kong (CN)

(72) Inventors: Zhaoxiang Bian, Hong Kong (CN); Lixiang Zhai, Hong Kong (CN); Lin Zhu, Hong Kong (CN); Yaqi Li, Hong Kong (CN); Yujuan Zhou, Hong Kong (CN); Mengyang Hou, Hong Kong (CN); Chengyuan Lin, Hong Kong (CN)

(21) Appl. No.: 19/275,270

(22) Filed: Jul. 21, 2025

Related U.S. Application Data
(60) Provisional application No. 63/673,210, filed on Jul. 19, 2024.

(30) Foreign Application Priority Data
Mar. 5, 2025 (CN) 202510254625.9

(51) Int. Cl. A61K 36/505; A61K 31/05; A61K 31/4375; A61K 31/704; A61K 31/7048; A61K 36/48; A61K 36/575; A61K 36/65; A61P 1/10

(52) U.S. Cl. CPC A61K 36/505; A61K 31/05; A61K 31/4375; A61K 31/704; A61K 31/7048; A61K 36/48; A61K 36/575; A61K 36/65; A61P 1/10; A61K 2236/331; A61K 2236/333; A61K 2236/35; A61K 2236/39; A61K 2236/51

(57) ABSTRACT

A composition of traditional Chinese medicine (TCM) comprising Corydalis Rhizoma, Paeoniae Radix, Magnoliae Officinalis Cortex, and Sennae Folium; a composition of extracts of the TCM composition; a pharmaceutical composition thereof; a method of using the same for treating constipation, including constipation-predominant symptoms (IBS-C); and a method of preparing the compositions. The various compositions disclosed herein are safe and effective in treating constipations.

### CROSS-REFERENCE TO RELATED APPLICATIONS

The present application claims priority to U.S. Provisional Patent Application No. 63/673,210, filed on Jul. 19, 2024, and to Chinese application No. 202510254625.9, filed on Mar. 5, 2025. The contents of these applications are hereby incorporated by reference in their entirety.

### TECHNICAL FIELD

The present disclosure relates to traditional Chinese medicine compositions, including concentrated Chinese medicine granular compositions, purified Chinese medicine extraction compositions, Q-markers mixture compositions, or the like, methods of using the same for treating constipation, such as irritable bowel syndrome with constipation (IBS-C), as well as preparation methods thereof.

### BACKGROUND

Constipation, including chronic constipation, affects multiple clinical disciplines, significantly disrupts daily life, and impairs quality of life. It is a common and frequently encountered condition in clinical practice, typically characterized by infrequent bowel movements (fewer than three times per week), dry and hard stools that are difficult to pass, or defecation disorders even when stool consistency is normal. Defecation disorders may present as straining during defecation, difficulty in stool expulsion, a sensation of incomplete evacuation, prolonged defecation time, or the need for manual assistance to facilitate defecation. If symptoms persist for more than six months, the condition may be diagnosed as chronic constipation.

Abdominal pain refers to pain in the abdominal region caused by various conditions affecting intra-abdominal or extra-abdominal organs. The etiologies of acute and chronic abdominal pain are diverse, including diseases of abdominal organs, abdominal wall disorders, thoracic pathologies, poisoning, metabolic disturbances, organic neurological disorders, and systemic diseases. Constipation accompanied by severe abdominal pain, bloating, and vomiting may indicates acute constipation, which is often characterized by intestinal obstruction and stasis.

Irritable Bowel Syndrome (IBS) is a functional disorder characterized primarily by abdominal pain and alterations in bowel habits. Globally, the prevalence of IBS is more than 11%, with approximately one-third of IBS patients experiencing constipation-predominant symptoms (IBS-C), which are characterized by abdominal pain, reduced bowel frequency, and difficulty in passing stools. Clinical management involves lifestyle adjustments and medications.

Certain ingredients found in medicinal materials such as lignans, glycosides, alkaloids, and the like, may help promote bowel movements and relieve abdominal pain. These compounds may exert effects through mechanisms such as stimulating intestinal motility, regulating water absorption, or modulating neurotransmitter activity. However, the formulation of traditional Chinese medicine (TCM) prescriptions for specific conditions like constipation must adhere to the principles of TCM, including syndrome differentiation and individualized treatment strategies. Despite the availability of various therapeutic options, constipation remains a common and challenging condition. Therefore, there is an urgent need to develop novel therapeutic agents that are both effective and safe.

### SUMMARY

In a first aspect, provided herein is a traditional Chinese medicine composition comprising by dry weight: 1 to 25 parts of Corydalis Rhizoma; 5 to 50 parts of Paeoniae Radix; 1 to 30 parts of Magnoliae Officinalis Cortex; and 1 to 20 parts of Sennae Folium, wherein the Paeoniae Radix is Paeoniae Radix Rubra, Paeoniae Radix Alba, or a combination thereof, and wherein the Corydalis Rhizoma is Yanhusuo, Cuyanhusuo, or a combination thereof.

In certain embodiments, the traditional Chinese medicine composition comprises by dry weight: 5 to 15 parts of Corydalis Rhizoma; 15 to 25 parts of Paeoniae Radix; 10 to 20 parts of Magnoliae Officinalis Cortex; and 1 to 10 parts of Sennae Folium.

In certain embodiments, the traditional Chinese medicine composition comprises by dry weight: 5 to 10 parts of Corydalis Rhizoma; 15 to 20 parts of Paeoniae Radix; 10 to 15 parts of Magnoliae Officinalis Cortex; and 1 to 2 parts of Sennae Folium.

In certain embodiments, the traditional Chinese medicine composition comprises by dry weight: 5 or 10 parts of Corydalis Rhizoma; 15 or 20 parts of e Paeoniae Radix; 10 or 15 parts of Magnoliae Officinalis Cortex; and 1 or 2 parts of Sennae Folium.

In certain embodiments, the traditional Chinese medicine composition comprises: 0.1449 mg/mL sennoside B, 0.1436 mg/mL magnolol, 2.7389 mg/mL paeoniflorin, and 0.0112 mg/mL tetrahydropalmatine.

In certain embodiments, the traditional Chinese medicine composition is in a form selected from the group consisting of a solid mixture, a capsule, a tablet, a powder, a granule, and a liquid formulation.

In a second aspect, provided herein is a composition comprising by weight: 1-10% sennoside A, 1-10% sennoside B, 1-10% magnolol, 1-10% honokiol, 30-50% paeoniflorin, 5-10% albiflorin, 0.1-0.5% corydaline, and 0.1-0.5% tetrahydropalmatine.

In certain embodiments, the composition comprises by weight: 1-5% sennoside A, 1-5% sennoside B, 1-5% magnolol, 1-5% honokiol, 30-50% paeoniflorin, 5-10% albiflorin, 0.1-0.3% corydaline, and 0.1-0.3% tetrahydropalmatine.

In certain embodiments, the composition comprises by weight: 1-2% sennoside A, 1-3% sennoside B, 1-3% magnolol, 1-2% honokiol, 40-45% paeoniflorin, 5-7% albiflorin, 0.1-0.2% corydaline, and 0.1-0.2% tetrahydropalmatine.

In certain embodiments, the composition is obtained from a traditional Chinese medicine composition comprising: Corydalis Rhizoma; Paeoniae Radix; Magnoliae Officinalis Cortex; and Sennae Folium, wherein the Paeoniae Radix is Paeoniae Radix Rubra, Paeoniae Radix Alba, or a combination thereof, and wherein the Corydalis Rhizoma is Yanhusuo, Cuyanhusuo, or a combination thereof.

In certain embodiments, the composition comprises a solvent selected from water and 0.5% carboxymethylcellulose sodium (CMC-Na) aqueous solution.

In certain embodiments, the composition comprises: 0.0725-0.2899 mg/mL sennoside B, 0.0718-0.2872 mg/mL magnolol, 1.3695-5.4778 mg/mL paeoniflorin, and 0.0056-0.0225 mg/mL tetrahydropalmatine.

In a third aspect, provided herein is a method of treating constipation in a subject in need thereof, the method comprising: administering a therapeutically effective amount of the composition of any of the first aspect of the present invention to the subject.

In certain embodiments, the constipation is irritable bowel syndrome with constipation (IBS-C), slow transit constipation, outlet obstruction constipation, mixed-type constipation, drug-induced constipation, secondary constipation, or functional constipation; or wherein the constipation is accompanied by abdominal pain.

In a fourth aspect, provided herein is a method of treating constipation in a subject in need thereof, the method comprising: administering a therapeutically effective amount of the composition of any of the second aspect of the present disclosure to the subject.

In certain embodiments, the constipation is irritable bowel syndrome with constipation (IBS-C), slow transit constipation, outlet obstruction constipation, mixed-type constipation, drug-induced constipation, secondary constipation, or functional constipation; or wherein the constipation is accompanied by abdominal pain.

In a fifth aspect, provided herein is a method of preparing the composition of any of the second aspect of the present disclosure, the method comprising: combining Corydalis Rhizoma; Paeoniae Radix; Magnoliae Officinalis Cortex; and Sennae Folium, in a mass ratio of (5-15):(15-25):(10-20):(1:10) respectively to form a mixture, wherein the Paeoniae Radix is Paeoniae Radix Rubra, Paeoniae Radix Alba, or a combination thereof, and wherein the Corydalis Rhizoma is Yanhusuo, Cuyanhusuo, or a combination thereof; and extracting sennoside A, sennoside B, magnolol, honokiol, paeoniflorin, albiflorin, corydaline, and tetrahydropalmatine from the raw materials to obtain the composition.

### BRIEF DESCRIPTION OF DRAWINGS

FIG. 1 shows a standard curve of magnolol according to an Example of the present disclosure.

FIG. 2 shows a standard curve of honokiol according to an Example of the present disclosure.

FIG. 3 shows a standard curve of sennoside A according to an Example of the present disclosure.

FIG. 4 shows a standard curve of sennoside B according to an Example of the present disclosure.

FIG. 5 shows the standard curve of paeoniflorin according to an Example of the present disclosure.

FIG. 6 shows the standard curve of albiflorin according to an Example of the present disclosure.

FIG. 7 shows the standard curve of tetrahydropalmatine according to an Example of the present disclosure.

FIG. 8 shows the standard curve of corydaline according to an Example of the present disclosure.

FIG. 9 shows the effect of four optional CCMG formulations of CDD-2105 on the stool frequency within 6 h of loperamide-induced constipation in mice.

FIG. 10 shows the effect of four optional CCMG formulations of CDD-2105 on the fecal water content of mice with loperamide-induced constipation.

FIG. 11 shows the effect of the PCME formulations of CDD-2105 on the stool frequency within 6 h in loperamide-induced constipated mice.

FIG. 12 shows the effect of the PCME formulations of CDD-2105 on the fecal water content of mice with loperamide-induced constipation.

FIG. 13 shows the effect of four optional CCMG compositions of CDD-2105 on the latency of heat-stimulation-induced nociception in mice

FIG. 14 shows the effect of the PCME formulations of CDD-2105 on the latency of heat-stimulation-induced nociception in mice.

FIG. 15 shows the effect of the Q-Marker Mixture formulations of CDD-2105 on the body weight of mice.

FIG. 16 shows the effect of the Q-Marker Mixture formulations of CDD-2105 on the food intake of mice.

FIG. 17 shows the effect of the Q-Marker Mixture formulations of CDD-2105 on the organ coefficient of mice.

### DETAILED DESCRIPTION

### Definitions

Throughout the present specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the present invention.

Furthermore, throughout the present specification and claims, unless the context requires otherwise, the word “include” or variations such as “includes” or “including”, will be understood to imply the inclusion of a stated integer or group of integers but not the exclusion of any other integer or group of integers.

As used herein, the terms “treat”, “treating”, “treatment”, and the like refer to reducing or ameliorating a disorder/disease and/or symptoms associated therewith. It will be appreciated, although not precluded, treating a disorder or condition does not require that the disorder, condition, or symptoms associated therewith be completely eliminated. In certain embodiments, treatment includes prevention of a disorder or condition, and/or symptoms associated therewith. The term “prevention” or “prevent” as used herein refers to any action that inhibits or at least delays the development of a disorder, condition, or symptoms associated therewith. Prevention can include primary, secondary and tertiary prevention levels, wherein: a) primary prevention avoids the development of a disease; b) secondary prevention activities are aimed at early disease treatment, thereby increasing opportunities for interventions to prevent progression of the disease and emergence of symptoms; and c) tertiary prevention reduces the negative impact of an already established disease by restoring function and reducing disease-related complications.

The term “subject” as used herein, refers to an animal, typically a mammal or a human, that will be or has been the object of treatment, observation, and/or experiment. When the term is used in conjunction with administration of a composition/formulation/compound described herein, then the subject has been the object of treatment, observation, and/or administration of the composition, formulation or compound described herein.

The term “therapeutically effective amount” as used herein, means that amount of the composition, formulation, compound or pharmaceutical agent that elicits a biological and/or medicinal response in a cell culture, tissue system, subject, animal, or human that is being sought by a researcher, veterinarian, clinician, or physician, which includes alleviation of the symptoms of the disease, condition, or disorder being treated.

As used herein, the term “extract” or “extracts” in connection with a compound/ingredient described herein means the compound/ingredient is separated from at least a portion of the other components of a natural source, such as a raw material, e.g., a plant part.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±10%, ±7%, ±5%, ±3%, ±1%, or ±0% variation from the nominal value unless otherwise indicated or inferred.

As used herein, the term “raw material” or “raw materials” in the context of a traditional Chinese medicine (TCM) composition described herein, refers to a plant or a specific part of a plant (e.g., roots, leaves, bark, stems, branches, or tubers) obtained from the source plant. Unless otherwise specified, the term also encompasses plant materials that have undergone preliminary processing or treatment—such as chopping, sun-drying, or steaming—for the purpose of enhancing stability, extractability, or ease of administration in pharmaceutical or therapeutic applications.

As used herein, the term “a composition of traditional Chinese medicine (TCM)”, “a TCM composition” or similar expressions intend to encompass a composition comprising or consisting of the raw materials of the TCM, a composition comprising or consisting of Concentrated Chinese Medicine Granules (CCMG) of the TCM, a composition comprising or consisting of the extracts of the raw materials of the TCM (such as a PCME composition), a composition comprising or consisting of quality markers (Q-markers) of the raw materials of the TCM (either derived from the raw materials, synthesized, or obtained otherwise), unless specified otherwise. In certain embodiments, the above-mentioned composition may further comprise a solvent, thereby enabling the composition to be formulated in solution, dispersion, or suspension form.

The details of medicinal materials that can be comprised in or used to form the TCM composition of the present disclosure are listed in the following Table 1.

TABLE 1
Information of medicinal materials

| Chinese | English | | |
|---|---|---|---|
| pharmaceutical | pharmaceutical | | Medical use |
| name (Pinyin) | name | Source | part |
| Yanhusuo or | Corydalis Rhizoma | Corydalis yanhusuo | Dry tuber |
| Yuanhu | | W. T. Wang | |
| Cuyanhusuo | Corydalis Rhizoma | Corydalis yanhusuo | Dry tuber (made |
| | | W. T. Wang | with vinegar) |
| Baishao | Paeoniae Radix Alba | Paeonia lactiflora Pall. | Dry root |
| Chishao | Paeoniae Radix | Paeonia lactiflora Pall. or | Dry root |
| | Rubra | Paeonia veitchii Lynch | |
| Houpo | Magnoliae Officinalis | Magnolia officinalis Rehd. et | Dry tem bark, |
| | Cortex | Wils. or Magnolia officinalis | root bark or |
| | | Rehd. et Wils. var. biloba | branch bark |
| | | Rehd. et Wils. | |
| Fanxieye | Sennae Folium | Cassia angustifolia Vahl or | Dry leaflet |
| | | Cassia acutifolia Delile | |

As used herein, the term “a composition of Concentrated Chinese Medicine Granules (CCMG)”, “a composition/formulation of CCMG”, “a CCMG composition/formulation”, and “a granular formulation” in connection with a TCM composition described herein may be used interchangeably unless otherwise specified.

As used herein, the term “a composition of Purified Chinese Medicine Extracts (PCME)”, “a composition/formulation of PCME”, “a PCME composition/formulation”, and “an extraction formulation” in connection with a TCM composition described herein may be used interchangeably unless otherwise specified.

As used herein, the term “Cuyanhusuo” refers to vinegar-processed Corydalis Rhizoma, which has been subjected to vinegar processing (typically involving moistening with rice vinegar followed by stir-frying or steaming), for purposes such as enhancing the herb's analgesic and blood-activating properties, improving its pharmacological efficacy, and/or reducing potential toxicity.

In a first aspect, provided herein is a traditional Chinese medicine (TCM) composition comprising Corydalis Rhizoma, Paeoniae Radix, Magnoliae Officinalis Cortex, and Sennae Folium.

In certain embodiments, the Paeoniae Radix is Paeoniae Radix Rubra, Paeoniae Radix Alba, or a combination thereof.

In certain embodiments, the Corydalis Rhizoma is Yanhusuo, Cuyanhusuo, or a combination thereof.

In certain embodiments, the TCM composition comprises by dry weight: 1 to 25 parts of the Corydalis Rhizoma; 5 to 50 parts of the Paeoniae Radix; 1 to 30 parts of the Magnoliae Officinalis Cortex; and 1 to 20 parts of the Sennae Folium. In certain embodiments, the TCM composition comprises by dry weight: 5 to 15 parts of the Corydalis Rhizoma; 15 to 25 parts of the Paeoniae Radix; 10 to 20 parts of the Magnoliae Officinalis Cortex; and 1 to 10 parts of the Sennae Folium. In certain embodiments, the TCM composition comprises by dry weight: 5 to 10 parts of the Corydalis Rhizoma; 15 to 20 parts of the Paeoniae Radix; 10 to 15 parts of the Magnoliae Officinalis Cortex; and 1 to 2 parts of the Sennae Folium.

In certain embodiments, the TCM composition comprises by dry weight: 5 parts of the Corydalis Rhizoma; 15 parts of the Paeoniae Radix; 10 parts of the Magnoliae Officinalis Cortex; and 1 parts of the Sennae Folium. In certain embodiments, the TCM composition comprises by dry weight: 10 parts of the Corydalis Rhizoma; 20 parts of the Paeoniae Radix; 15 parts of the Magnoliae Officinalis Cortex; and 2 parts of the Sennae Folium. In certain embodiments, the TCM composition comprises by dry weight: 15 parts of the Corydalis Rhizoma; 25 parts of the Paeoniae Radix; 20 parts of the Magnoliae Officinalis Cortex; and 10 parts of the Sennae Folium. In certain embodiments, the TCM composition comprises by dry weight: 10 parts of the Corydalis Rhizoma; 20 parts of the Paeoniae Radix; 15 parts of the Magnoliae Officinalis Cortex; and 8 parts of the Sennae Folium. In certain embodiments, the Paeoniae Radix is Paeoniae Radix Rubra.

In certain embodiments, the TCM composition is a CDD-2105 formulation described in the examples of the present disclosure.

In certain embodiments, the traditional Chinese medicine composition comprises: 0.1449 mg/mL sennoside B, 0.1436 mg/mL magnolol, 2.7389 mg/mL paconiflorin, and 0.0112 mg/mL tetrahydropalmatine (THP).

In certain embodiments, the traditional Chinese medicine composition is in a form selected from the group consisting of a solid mixture, a capsule, a tablet, a powder, a granule, and a liquid formulation.

In a second aspect, provided herein is a composition comprising sennoside A, sennoside B, magnolol, honokiol, paeoniflorin, albiflorin, corydaline, and tetrahydropalmatine (THP).

In certain embodiments, the composition comprises by weight: 1-10% sennoside A, 1-10% sennoside B, 1-10% magnolol, 1-10% honokiol, 30-50% paeoniflorin, 5-10% albiflorin, 0.1-0.5% corydaline, and 0.1-0.5% tetrahydropalmatine.

In certain embodiments, the composition comprises by weight: 1-5% sennoside A, 1-5% sennoside B, 1-5% magnolol, 1-5% honokiol, 30-50% paeoniflorin, 5-10% albiflorin, 0.1-0.3% corydaline, and 0.1-0.3% tetrahydropalmatine.

In certain embodiments, the composition comprises by weight: 1-2% sennoside A, 1-3% sennoside B, 1-3% magnolol, 1-2% honokiol, 40-45% paeoniflorin, 5-7% albiflorin, 0.1-0.2% corydaline, and 0.1-0.2% tetrahydropalmatine.

In certain embodiments, the composition comprises by weight: 1.6539% sennoside A, 2.2611% sennoside B, 2.2408% magnolol, 1.6849% honokiol, 42.7401% paeoniflorin, 6.3684% albiflorin, 0.1748% corydaline, and 0.1407% tetrahydropalmatine.

In certain embodiments, the composition comprises sennoside A, sennoside B, magnolol, honokiol, paeoniflorin, albiflorin, corydaline, and tetrahydropalmatine in the amount listed in Table 2 below.

TABLE 2
Amounts of Marker compounds in the composition

| Marker compound | Content (wt %) |
|---|---|
| Sennoside A | 1.6539 |
| Sennoside B | 2.2611 |
| Magnolol | 2.2408 |
| Honokiol | 1.6849 |
| Paeoniflorin | 42.7401 |
| albiflorin | 6.3684 |
| Tetrahydropalmatine | 0.1748 |
| Corydaline | 0.1407 |

In certain embodiments, the composition is a CCMG composition. In certain embodiments, the composition is a PCME composition. In certain embodiments, the CCMG composition or PCME composition can be prepared from the medicinal materials shown in Table 1.

In certain embodiments, the composition comprises a solvent. In certain embodiments, the solvent is selected from the group consisting of water, and carboxymethylcellulose sodium (CMC-Na), such as 0.5% carboxymethylcellulose sodium (CMC-Na) aqueous solution.

In certain embodiments, the composition is obtained from a traditional Chinese medicine composition as described herein. In certain embodiments, the composition is obtained from a TCM composition comprising: Corydalis Rhizoma; Paeoniae Radix; Magnoliae Officinalis Cortex; and Sennae Folium. In certain embodiments, the Paeoniae Radix is Paeoniae Radix Rubra, Paeoniae Radix Alba, or a combination thereof. In certain embodiments, the Corydalis Rhizoma is Yanhusuo, Cuyanhusuo, or a combination thereof.

In certain embodiments, the composition is in a form selected from the group consisting of a solid mixture, a capsule, a tablet, a powder, a granule, and a liquid formulation (e.g., a solution, dispersion, or suspension).

In a third aspect, provided herein is a composition comprising sennoside A, sennoside B, magnolol, honokiol, paeoniflorin, albiflorin, corydaline, and tetrahydropalmatine (THP).

In certain embodiments, the composition comprises: 2.25-22.56 mg (e.g., 2.25-4.50 mg) of sennoside A, 2.32-23.18 mg (e.g., 2.32-4.64 mg) of sennoside B, 186.33-372.67 mg (e.g., 186.33-279.50 mg) of magnolol, 212.21-424.42 mg (e.g., 212.21-318.32 mg) of honokiol, 480.15-800.26 mg (e.g., 480.15-640.20 mg) of paeoniflorin, 257.49-429.15 mg (e.g., 257.49-343.32 mg) of albiflorin, 0.75-2.25 mg (e.g., 0.75-1.50 mg) of corydaline, and 0.61-1.84 mg (e.g., 0.61-1.23 mg) of tetrahydropalmatine.

In certain embodiments, the composition comprises 4-5 mg of sennoside A, 4-5 mg of sennoside B, 250-300 mg of magnolol, 300-330 mg of honokiol, 620-660 mg of paeoniflorin, 320-360 mg of albiflorin, 1-5 mg of corydaline, and 1-5 mg of tetrahydropalmatine. In certain embodiments, the composition comprises 4.50 mg of sennoside A, 4.64 mg of sennoside B, 279.50 mg of magnolol, 318.32 mg of honokiol, 640.20 mg of paeoniflorin, 343.32 mg of albiflorin, 1.50 mg of corydaline, and 1.23 mg of tetrahydropalmatine.

In certain embodiments, the composition comprises sennoside A, sennoside B, magnolol, honokiol, paeoniflorin, albiflorin, corydaline, and tetrahydropalmatine in the amounts listed in any formulation of below Table 3.

TABLE 3
Amounts of Marker compounds in the composition

| | Content (mg) | | | |
|---|---|---|---|---|
| | THP/ | Paeoniflorin/ | Magnolol/ | Sennoside A/ |
| Formulation | Corydaline | Albiflorin | Honokiol | Sennoside B |
| Formulation | 0.61/ | 480.15/ | 186.33/ | 2.25/ |
| CCMG_1 | 0.75 | 257.49 | 212.21 | 2.32 |
| Formulation | 1.84/ | 800.26/ | 372.67/ | 22.56/ |
| CCMG_2 | 2.25 | 429.15 | 424.42 | 23.18 |
| Formulation | 1.23/ | 640.20/ | 279.50/ | 18.01/ |
| CCMG_3 | 1.50 | 343.32 | 318.32 | 18.54 |
| Formulation | 1.23/ | 640.20/ | 279.50/ | 4.50/ |
| CCMG_4 | 1.50 | 343.32 | 318.32 | 4.64 |

In certain embodiments, the composition is a CCMG composition. In certain embodiments, the composition is a PCME composition. In certain embodiments, the CCMG composition or PCME composition can be prepared from the medicinal materials shown in Table 1.

In certain embodiments, the formulations in Table 3 can be obtained from the TCM composition of any aspects (such as the first aspect) of the present disclosure.

In certain embodiments, the composition comprises a solvent. In certain embodiments, the solvent is selected from water and 0.5% carboxymethylcellulose sodium (CMC-Na) aqueous solution.

In certain embodiments, the composition comprises: 0.05-0.5 mg/mL sennoside B, 0.05-0.5 mg/mL magnolol, 1.0-8 mg/mL paeoniflorin, and 0.004-0.05 mg/mL tetrahydropalmatine. In certain embodiments, the composition comprises: 0.07-0.30 mg/mL sennoside B, 0.07-0.30 mg/mL magnolol, 1.30-5.50 mg/mL paeoniflorin, and 0.005-0.030 mg/mL tetrahydropalmatine. In certain embodiments, the composition comprises 0.0725-0.2899 mg/mL sennoside B, 0.0718-0.2872 mg/mL magnolol, 1.3695-5.4778 mg/mL paeoniflorin, and 0.0056-0.0225 mg/ml tetrahydropalmatine. In certain embodiments, the composition comprises: 0.0725 mg/ml sennoside B, 0.0718 mg/mL magnolol, 1.3695 mg/mL paeoniflorin, and 0.0056 mg/mL tetrahydropalmatine. In certain embodiments, the composition comprises: 0.15 mg/mL sennoside B, 0.15 mg/mL magnolol, 2.7 mg/mL paeoniflorin, and 0.01 mg/mL tetrahydropalmatine. In certain embodiments, the composition comprises: 0.2899 mg/mL sennoside B, 0.2872 mg/mL magnolol, 5.4778 mg/mL paconiflorin, and 0.0225 mg/mL tetrahydropalmatine.

In certain embodiments, the composition is obtained from a traditional Chinese medicine composition comprising: Corydalis Rhizoma; Paeoniae Radix; Magnoliae Officinalis Cortex; and Sennae Folium. In certain embodiments, the Paeoniae Radix is Paeoniae Radix Rubra, Paeoniae Radix Alba, or a combination thereof. In certain embodiments, the Corydalis Rhizoma is Yanhusuo, Cuyanhusuo, or a combination thereof.

In certain embodiments, the composition comprises extracts of the TCM composition of any aspects described herein.

In certain embodiments, the composition is in a form selected from the group consisting of a solid mixture, a capsule, a tablet, a powder, a granule, and a liquid formulation (e.g., a solution, dispersion, or suspension).

In a fourth aspect, provided herein is a method of treating constipation in a subject in need thereof, the method comprising: administering a therapeutically effective amount of the composition of any aspects described herein to the subject.

In certain embodiments, the constipation is irritable bowel syndrome with constipation (IBS-C), slow transit constipation, outlet obstruction constipation, mixed-type constipation, drug-induced constipation, secondary constipation, or functional constipation; or wherein the constipation is accompanied by abdominal pain.

In certain embodiments, the subject is a human, a non-human primate, a rodent, a canine, a feline, a bovine, or an equine.

In a sixth aspect, provided herein is a method of preparing the composition of any aspects of the present disclosure, the method comprising: combining Corydalis Rhizoma; Paeoniae Radix; Magnoliae Officinalis Cortex; and Sennae Folium to form a mixture, and extracting sennoside A, sennoside B, magnolol, honokiol, paeoniflorin, albiflorin, corydaline, and tetrahydropalmatine from the raw materials to obtain the composition.

In certain embodiments, the Corydalis Rhizoma; Paeoniae Radix; Magnoliae Officinalis Cortex; and Sennae Folium is in a mass ratio of (5-15):(15-25):(10-20):(1:10).

In certain embodiments, the Paeoniae Radix is Paeoniae Radix Rubra, Paeoniae Radix Alba, or a combination thereof. In certain embodiments, the Corydalis Rhizoma is Yanhusuo, Cuyanhusuo, or a combination thereof.

In a seventh aspect, provided herein is a method of preparing the traditional Chinese medicine composition of any aspects of the present disclsoure, comprising: combining the Corydalis Rhizoma; Paeoniae Radix; Magnoliae Officinalis Cortex; and Sennae Folium to form a mixture; decocting the mixture in water to obtain a medicinal decoction.

In certain embodiments, the method further comprises concentrating and drying the medicinal decoction to obtain concentrated Chinese medicine granules.

In certain embodiments, the Paeoniae Radix is Paeoniae Radix Rubra, Paeoniae Radix Alba, or a combination thereof. In certain embodiments, the Corydalis Rhizoma is Yanhusuo, Cuyanhusuo, or a combination thereof.

In certain embodiments, the Paeoniae Radix is Paeoniae Radix Rubra, Paeoniae Radix Alba, or a combination thereof. In certain embodiments, the Corydalis Rhizoma is Yanhusuo, Cuyanhusuo, or a combination thereof.

In a further aspect, provided herein is a composition of traditional Chinese medicine (TCM) extracts comprising, based on 100% of the dry matter content of the composition: 1-10% sennoside A, 1-10% sennoside B, 1-10% magnolol, 1-10% honokiol, 30-50% paeoniflorin, 5-10% albiflorin, 0.1-0.5% corydaline, and 0.1-0.5% tetrahydropalmatine. In certain embodiments, the remainder comprises other substances.

In certain embodiments, the composition of TCM extracts comprises, based on 100% of the dry matter content of the composition: 1-5% sennoside A, 1-5% sennoside B, 1-5% magnolol, 1-5% honokiol, 30-50% paeoniflorin, 5-10% albiflorin, 0.1-0.3% corydaline, and 0.1-0.3% tetrahydropalmatine. In certain embodiments, the balance is other substances. In certain embodiments, the remainder comprises other substances.

In certain embodiments, the composition of TCM extracts comprises, based on 100% of the dry matter content of the composition: 1-3% sennoside A, 1-3% sennoside B, 1-3% magnolol, 1-3% honokiol, 35-50% paeoniflorin, 5-10% albiflorin, 0.1-0.3% corydaline, and 0.1-0.3% tetrahydropalmatine. In certain embodiments, the remainder comprises other substances.

In a further aspect, provided herein is a composition of traditional Chinese medicine (TCM) extracts comprising extracts from raw materials comprising: Magnoliae Officinalis Cortex; Sennae Folium; Paeoniae Radix Rubra or Paeoniae Radix Alba; and Corydalis Rhizoma.

In certain embodiments, the Corydalis Rhizoma is Yanhusuo or Cuyanhusuo.

In certain embodiments, the weight ratio of the raw materials (i.e., Magnoliae Officinalis Cortex; Sennae Folium; Paeoniae Radix Rubra or Paeoniae Radix Alba; and Yanhusuo or Cuyanhusuo) is (1-20):(1-30):(1-25):(5-50). In certain embodiments, the weight ratio of Magnoliae Officinalis Cortex: Sennae Folium: Paeoniae Radix Rubra or Paeoniae Radix Alba: Yanhusuo or Cuyanhusuo is (10-20):(1-10):(15-25):(5-15). In certain embodiments, the weight ratio of Magnoliae Officinalis Cortex: Sennae Folium: Paeoniae Radix Rubra or Paeoniae Radix Alba: Yanhusuo or Cuyanhusuo is (1-20):(1-10):(20-50):(1-10). In certain embodiments, the weight ratio of Magnoliae Officinalis Cortex: Sennae Folium: Paeoniae Radix Rubra or PaeoniaeRadix Alba: Yanhusuo or Cuyanhusuo is (10-20):(1-5):(15-25):(8-15). In certain embodiments, the weight ratio of Magnoliae Officinalis Cortex: Sennae Folium: Paeoniae Radix Rubra or Paeoniae Radix Alba: Yanhusuo or Cuyanhusuo is 15:2:20:10.

In certain embodiments, the composition described in the present disclosure can be composed of Concentrated Chinese Medicine Granules (CCMG) containing equivalent amounts of the raw materials of the TCM compositions as described herein.

In certain embodiments, the composition described in the present disclosure can be composed of Purified Chinese Medicine Extracts (PCME) containing quality markers (Q-markers) of the same amount of the raw materials of the TCM compositions as described herein.

In a further aspect, provided herein is a pharmaceutical composition for treating constipation, comprising, consisting essentially of, or consisting of the composition of TCM extracts described in the present disclosure and one or more pharmaceutically acceptable excipients.

In certain embodiments, composition of any aspects of the present disclosure or the pharmaceutical composition has a dosage form selected from one or two, or more of the group consisting of a capsule, a tablet, a powder, a solution, and a suspension.

In a further aspect, provided herein is a method for preparing the TCM extract composition according to any aspect of the present disclosure or the pharmaceutical composition described herein, comprising: combining the extracts of Magnoliae Officinalis Cortex; Sennae Folium; Paeoniae Radix Rubra or Paeoniae Radix Alba; and Yanhusuo or Cuyanhusuo to obtain the composition or pharmaceutical composition.

In certain embodiments, the extract of Magnoliae Officinalis Cortex is prepared by extracting Magnoliae Officinalis Cortex with water, ethanol, and/or petroleum ether to obtain an extraction solution; concentrating the extraction solution to obtain an extractum; and extracting the extractum with petroleum ether. In certain embodiments, the extraction temperature with water or ethanol is 40° C. to 90° C. In certain embodiments, the extraction temperature with petroleum ether is 40° C. to 70° C.

In certain embodiments, the extract of Sennae Folium is prepared by extracting Sennae Folium with water, sodium bicarbonate, or ethanol to obtain an extraction solution; concentrating the extraction solution to obtain a concentrate; and purifying the concentrate by column chromatography to obtain the final extract. In certain embodiments, the extraction temperature with water, sodium bicarbonate, or ethanol is 25° C. to 90° C.

In certain embodiments, the extract of Paeoniae Radix Alba or Paeoniae Radix Rubra is prepared by extracting the material with water or ethanol to obtain an extraction solution; concentrating the extraction solution to obtain a concentrate; and purifying the concentrate by column chromatography to obtain the final extract. In certain embodiments, the extraction temperature with water or ethanol is 60° C. to 90° C.

In certain embodiments, the extract of Yanhusuo or Cuyanhusuo is prepared by extracting the material with water or ethanol to obtain an extraction solution; concentrating the extraction solution to obtain a concentrate; and purifying the concentrate by column chromatography to obtain the final extract. In certain embodiments, the extraction temperature with water or ethanol is 60° C. to 90° C.

In certain embodiments, the extract of Magnoliae Officinalis Cortex is prepared by the following steps: (1) pulverizing Magnoliae Officinalis Cortex, extracting with water or 30%-95% ethanol in 8-12 times the volume, and refluxing in a water bath at 40° C.-90° C. for 1-3 cycles, each lasting 0.5-1.5 hours, thereby obtaining extraction solutions; (2) filtering and combining the extraction solutions to obtain a filtrate, and concentrating the filtrate under reduced pressure at 40° C.-80° C. to yield an extractum with a relative density of 1.15-1.20; (3) adding 50-150 times the volume of petroleum ether to the extractum, extracting by water bath reflux at 40° C.-70° C. for 3-12 hours to obtain a petroleum ether extraction solution; and (4) cooling the petroleum ether extraction solution to induce crystallization, evaporating the petroleum ether at 40-70° C., and freeze-drying to obtain the final extract.

In certain embodiments, the extract of Sennae Folium is prepared by the following steps: (1) extracting Sennae Folium with water, 0.1% sodium bicarbonate, or 30%-95% ethanol in 80-120 times the volume, and refluxing in a water bath at 25° C.-90° C. for 1-3 cycles, each lasting 0.5-1.5 hours, to obtain extraction solutions; (2) filtering and combining the extraction solutions to obtain a filtrate, and concentrating the filtrate under reduced pressure at 40° C.-80° C. to yield a concentrate with a relative density of 1.05-1.10; and (3) purifying the concentrate by column chromatography, eluting with 5-10 column volumes of 5% ethanol to obtain an eluent, and freeze-drying the eluent to obtain the final extract.

In certain embodiments, the extract of Paeoniae Radix Alba or Paeoniae Radix Rubra is prepared by the following steps: (1) pulverizing the material, extracting with water or 30%-95% ethanol in 8-12 times the volume, and refluxing in a water bath at 60° C.-90° C. for 1-3 cycles, each lasting 0.5-1.5 hours, to obtain extraction solutions; (2) filtering and combining the extraction solutions to obtain a filtrate, and concentrating the filtrate under reduced pressure at 40° C.-80° C. to yield a concentrate with a relative density of 1.05-1.10; and (3) purifying the concentrate by column chromatography, eluting with 8-16 column volumes of 20% ethanol to obtain an eluent, and freeze-drying the eluent to obtain the final extract.

In certain embodiments, the extract of Yanhusuo or Cuyanhusuo is prepared by the following steps: (1) pulverizing the material, extracting with water or 30%-95% ethanol in 8-12 times the volume, and refluxing in a water bath at 60° C.-90° C. for 1-3 cycles, each lasting 0.5-1.5 hours, to obtain extraction solutions; (2) filtering and combining the extraction solutions to obtain a filtrate, and concentrating the filtrate under reduced pressure at 40° C.-80° C. to yield a concentrate with a relative density of 1.05-1.10; and (3) purifying the concentrate by column chromatography, eluting with 5-8 column volumes of 95% ethanol to obtain an eluent, and freeze-drying the eluent to obtain the final extract.

In certain embodiments, the preparation method further comprises a step of detecting the active ingredients in the composition of TCM extracts or the pharmaceutical composition. In certain embodiments, the active ingredients include: Magnolol, Honokiol, Sennoside A, Sennoside B, Paeoniflorin, Albiflorin, Tetrahydropalmatine, and Corydaline.

In certain embodiments, the detection step is performed using liquid chromatography-tandem mass spectrometry (LC-MS/MS).

In certain embodiments, the chromatographic conditions include: (i) Column: octadecylsilane-bonded silica gel as filler, with theoretical plate number ≥1000; (ii) Column temperature: 40° C.; (iii) Mobile phase: A (0.1% formic acid aqueous solution), B (0.1% formic acid acetonitrile solution); and (iv) Gradient elution, with the elution procedure shown in Table 4a below.

TABLE 4a
Gradient elution program

| Time (min) | Mobile phase A (%) | Mobile phase B (%) |
|---|---|---|
| 0-6 | 88→75 | 12→25 |
| 6-7 | 75→55 | 25→45 |
| 7-10 | 55→20 | 45→80 |
| 10-10.1 | 20→0 | 80→100 |

In certain embodiments, the mass spectrometry conditions include: a triple quadrupole mass spectrometer; electrospray ionization (ESI) in negative mode; a detection mode of multiple reaction monitoring (MRM); and MRM ion pairs specific to various active ingredients.

In certain embodiments, the MRM ion pairs are listed in Table 4b.

TABLE 4b
MRM ion pairs specific to various active ingredients.

| Ingredient | MRM ion pairs |
|---|---|
| Magnolol | m/z 265.0 → 247.1 and m/z 265.0 → 245.0 |
| Honokiol | m/z 265.0 → 249.0 and m/z 265.0 → 223.0 |
| Sennoside A | m/z 861.4 → 699.0 and m/z 861.4 → 386.0 |
| Sennoside B | m/z 861.4 → 699.0 and m/z 861.4 → 386.0 |
| Paeoniflorin | m/z 525.0 → 327.0 and m/z 525.0 → 121.0 |
| Albiflorin | m/z 525.0 → 479.0 and m/z 525.0 → 121.0 |
| Tetrahydropalmatine | m/z 356.1 → 159.0 and m/z 356.1 → 159.0 |
| Corydaline | m/z 370.2 → 170.0 and m/z 370.2 → 170.0 |

As demonstrated in the following examples, the TCM composition—whether in the form of a CCMG formulation, PCME formulation, MM formulation, or pharmaceutical composition—effectively alleviates constipation, including symptoms of IBS-C, without presenting risks of severe acute toxicity, thereby offering high utilization efficiency.

The present invention is further described in detail below through specific examples, which should not be construed as limiting the protection scope of the present invention.

### EXAMPLE A: PREPARATION OF PURIFIED CHINESE MEDICINE EXTRACTS (PCME) AND QUALITY CONTROL THEREOF

### 1. Instruments

Ultrasonic cleaner (KQ-500DE, Kunshan Ultrasonic Instrument Co., Ltd., Jiangsu, China); ultra-high performance liquid chromatography coupled triple quadrupole mass spectrometer (Agilent 1290 InfinityII-Agilent 6470 LC/TQ, Agilent Technologies, Inc., USA); electronic analytical balance (ml204/02, Mettler-Toledo International Inc., Columbus, Ohio, USA); electronic analytical balance (XPR205, Mettler-Toledo International Inc., Columbus, Ohio, USA); electronic analytical balance (E600-2, G & G Measurement Plant, Changshu, China).

### 2. Materials and Reagents

Raw materials Magnolia officinalis Cortex (Lot No.: 20221014-13), Sennae Folium (Lot No.: 20221014-02), Paeoniae Radix Alba (Lot No.: 20221014-07), Paeoniae Radix Rubra (Lot No.: 20221014-08), Corydalis Rhizoma (Lot No.: 20221014-10), all bought from Shanghai Content Worry-Free Standard Technology Service Co., Ltd. (Shanghai, China). Raw material Corydalis Rhizoma (Lot No.: 2210006-ZJ) was bought from Sichuan Neautus Traditional Chinese Medicine CO.,Ltd. (Sichuang, China).

Magnolol (Lot No.: 110729-202015), obtained from National Institutes for Food and Drug Control (Beijing, China); sennoside A (Lot No.: CFS202201) and sennoside B (Lot No.: CFS202102), obtained from Wuhan ChemFaces Biochemical Co., Ltd. (Wuhan, China); paconiflorin (Lot No.: NO811A), obtained from Dalian Meilun Biotech Co., Ltd. (Dalian, China); honokiol (Lot No.: YY90181), albiflorin (Lot No.: B21149), tetrahydropalmatine (Lot No.: B74569), and corydaline (Lot No.: B21425), all obtained from Shanghai yuanye Bio-Technology Co., Ltd. (Shanghai, China).

Acetonitrile, chromatographic grade, was purchased from Omnilab-Laborzentrum GmbH & Co. KG (Bremen, Germany); methanol, analytical reagent grade, was purchased from Guangdong Guanghua Sci-Tech Co., Ltd. (Guangdong, China); and ultrapure water was used.

### Example A-1: Extraction and Purification of the Raw Materials

### (1). Magnolia Officinalis Cortex

The process was conducted as follows: 15 g of Magnolia officinalis Cortex was pulverized (passed through a 100-mesh sieve), and extracted 3 times with 12 volumes of 95% ethanol under reflux in a water bath, each extraction lasting 1.5 hours. The extract solutions were filtered, combined, and concentrated under reduced pressure at 40-80° C. to obtain an extractum with a relative density of 1.15-1.20. The extractum was further extracted with 150 volumes of petroleum ether by refluxing in a 40° C. water bath for 12 hours. The petroleum ether extract was separated while hot, then cooled at 4° C. overnight to induce crystallization. The residual petroleum ether was evaporated at 40-70° C.

The resulting material was lyophilized to yield a dry extract powder of Magnolia officinalis Cortex, with a yield of 4.91% and a purity of 81.17%. The contents of magnolol and honokiol were 37.95% and 43.22%, respectively.

### (2) Sennae Folium

The process was conducted as follows: 2 g of unground Sennae Folium was extracted 3 times with 80 volumes of water under reflux in a water bath, cach extraction lasting 30 min. The extract solutions were filtered, combined, and concentrated under reduced pressure at 40-80° C. to obtain a concentrate with a relative density of 1.05-1.10. AB-8 macroporous resin was prepared and packed into a column, and the concentrate was loaded using the wet method. Impurities were removed using 5 column volumes of water and 8 column volumes of 0.5% NaHCO₃. Then the column was eluted with 10 column volumes of 5% ethanol. The eluate was collected and freeze-dried to yield a dry extract powder, with a yield of 1.53% and a purity of 29.87%. The contents of sennoside A and sennoside B were 14.71% and 15.15%, respectively.

### (3) Paeoniae Radix Alba

The process was conducted as follows: 20 g of Paeoniae Radix Alba was pulverized (passed through a 100-mesh sieve), and extracted 3 times with 12 volumes of 30% ethanol under reflux in a water bath, cach extraction lasting 30 min. The extract solutions were filtered, combined, and concentrated under reduced pressure at 40-80° C. to obtain a concentrate with a relative density of 1.05-1.10. AB-8 macroporous resin was prepared and packed into a column, and the concentrate was loaded using the wet method. Impurities were removed using 8 column volumes of water, followed by elution with 16 column volumes of 20% ethanol. The eluate was collected and freeze-dried to yield a dry extract powder, with a yield of 6% and a purity of 81.96%. The contents of paconiflorin and albiflorin were 53.35% and 28.61%, respectively.

### (4) Corydalis Rhizoma (Cuyanhusuo)

The process was conducted as follows: 10 g of cuyanhusuo was pulverized (passed through a 100-mesh sieve), and extracted 3 times with 10 volumes of 50% ethanol under reflux in a water bath, each extraction lasting 30 min. The extract solutions were filtered, combined, and concentrated under reduced pressure at 40-80° C. to obtain a concentrate with a relative density of 1.05-1.10. AB-8 macroporous resin was prepared and packed into a column, and the concentrate was loaded using the wet method. Impurities were removed using 8 column volumes of water and 8 column volumes of 40% ethanol. Then the column was eluted with 8 column volumes of 95% ethanol. The eluate was collected and freeze-dried to yield a dry extract powder, with a yield of 0.187% and a purity of 14.58%. The contents of tetrahydropalmatine and corydaline were 6.57% and 8.01%, respectively.

### (5) Preparation of PCME

20 mg, 69.83 mg, 374 mg, and 12.48 mg of the dry extract powders of Magnolia officinalis Cortex, Sennae Folium, Paeoniae Radix Alba, and Cuyanhusuo, respectively, as obtained above, were mixed to obtain a purified Chinese medicine extract composition (PCME).

### Example A-2: Quality Control of Marker Compounds in the PCME

The content and marker compounds in the PCME were determined using liquid chromatography-mass spectrometry (LC-MS/MS), as described below:

### 1. Chromatographic Conditions and System Suitability Test

A column packed with octadecylsilane-bonded silica gel was used as the stationary phase. The mobile phase consisted of 0.1% formic acid aqueous solution (A) and 0.1% formic acid in acetonitrile (B), with gradient elution performed according to the program shown in above Table 2.

### 2. Determination of Marker Compounds and Contents Thereof

### (1). Magnolol and Honokiol

The chromatographic conditions described under above section A (Chromatographic conditions and System Suitability Test) were used, with the column temperature maintained at 40° C. A triple quadrupole mass spectrometer equipped with an electrospray ionization source in negative mode (ESI⁻) was used. Multiple reaction monitoring (MRM) was performed using the ion pairs m/z 265.0→247.1 and m/z 265.0→245.0. The theoretical plate number of the column was not less than 1000, calculated with reference to the magnolol peak.

The column temperature was maintained at 40° C. A triple quadrupole mass spectrometer equipped with an electrospray ionization source in negative mode (ESI⁻) was used. MRM was performed using the ion pairs m/z 265.0→249.0 and m/z 265.0→223.0. The theoretical plate number of the column was not less than 1000, calculated with reference to the honokiol peak.

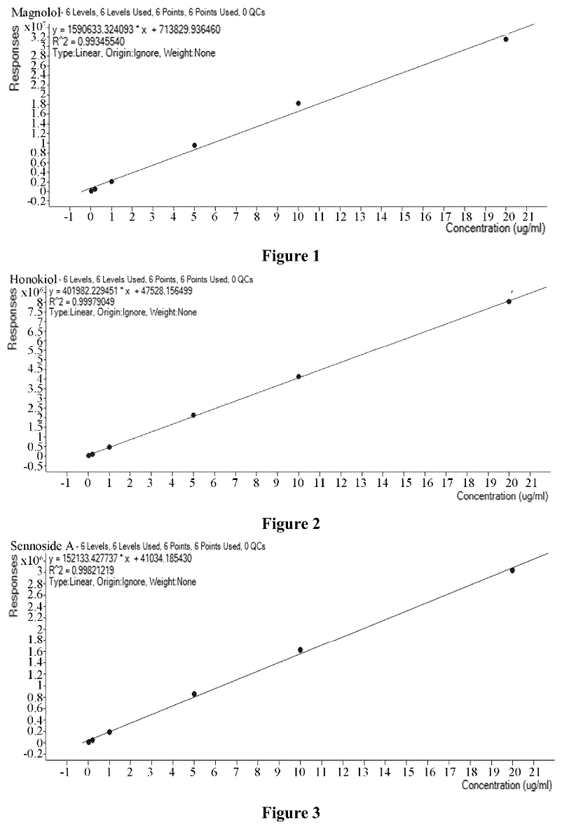

Preparation of reference solutions: appropriate amounts of magnolol and honokiol reference standards were accurately weighed and dissolved in methanol to prepare a series of standard solutions containing 6.0, 15.0, 30.0, 60.0, and 90.0 μg/mL of magnolol and honokiol, respectively. FIG. 1 shows the calibration curve of magnolol; FIG. 2 shows the calibration curve of honokiol.

Preparation of test solution: about 0.2 g of the PCME was accurately weighed and placed in a stoppered conical flask. Then, an accurate amount of 25 mL of methanol was added. The mixture was soaked for 24 hours and filtered. The subsequent filtrate was collected thereby obtaining the test solution.

Assay method: Precisely 1 μL each of the reference solution and the test solution was injected into the liquid chromatograph for determination, and the result was obtained accordingly.

### (2). Sennoside A and Sennoside B

The chromatographic conditions described under above section A (Chromatographic conditions and System Suitability Test) were used, with the column temperature maintained at 40° C. A triple quadrupole mass spectrometer equipped with an electrospray ionization source in negative mode (ESI⁻) was used. Multiple reaction monitoring (MRM) was performed using the ion pairs m/z 861.4→699.0 and m/z 861.4→386.0. The theoretical plate number of the column was not less than 1000, calculated with reference to the peaks of sennoside A and sennoside B.

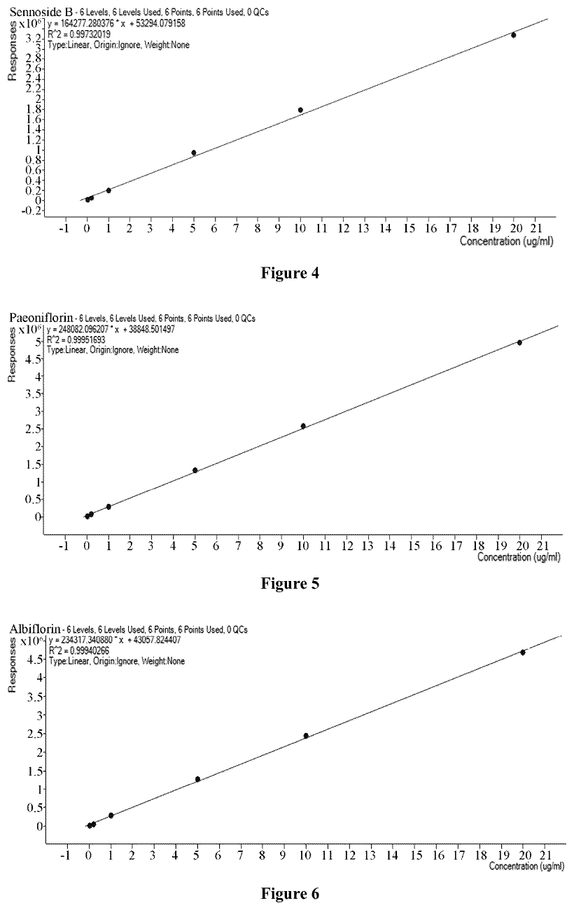

Preparation of reference solutions: appropriate amounts of sennoside A and sennoside B reference standards were accurately weighed and dissolved in 0.1% NaHCO₃ solution to prepare a series of standard solutions containing 6.0, 15.0, 30.0, 60.0, and 90.0 μg/mL of sennoside A and sennoside B, respectively, thereby obtaining the reference solutions. FIG. 3 shows the calibration curve of sennoside A; FIG. 4 shows the calibration curve of sennoside B.

Preparation of test solution: about 0.5 g of the PCME was accurately weighed and placed in a stoppered conical flask. Then, an accurate amount of 50 mL of 0.1% NaHCO₃ solution was added. The total weight was recorded. The mixture was sonicated (power: 250 W, frequency: 40 kHz) for 15 minutes. The flask was removed, cooled, and weighed again. The lost weight was replenished with 0.1% NaHCO₃ solution, shaken well, and filtered. The subsequent filtrate was collected thereby obtaining the test solution.

Assay method: Precisely 2 μL each of the reference solution and the test solution was injected into the liquid chromatograph for determination, and the result was obtained accordingly.

### (3). Paconiflorin and Albiflorin

The chromatographic conditions described under above section A (Chromatographic conditions and System Suitability Test) were used, with the column temperature maintained at 40° C. A triple quadrupole mass spectrometer equipped with an electrospray ionization source in negative mode (ESI⁻) was used. MRM was performed using the ion pairs m/z 525.0→327.0 and m/z 525.0→121.0. The theoretical plate number of the column was not less than 1000, calculated with reference to the paconiflorin peak.

The column temperature was maintained at 40° C. MRM was performed using the ion pairs m/z 525.0→479.0 and m/z 525.0→121.0. The theoretical plate number of the column was not less than 1000, calculated with reference to the albiflorin peak.

Preparation of reference solutions: appropriate amounts of paconiflorin and albiflorin reference standards were accurately weighed and dissolved in methanol to prepare a series of standard solutions containing 6.0, 15.0, 30.0, 60.0, and 90.0 μg/mL of each compound, thereby obtaining the reference solutions. FIG. 5 shows the calibration curve of paconiflorin; FIG. 6 shows the calibration curve of albiflorin.

Preparation of test solution: about 0.5 g of the PCME was accurately weighed and placed in a stoppered conical flask. Then, an accurate amount of 25 mL of methanol was added. The total weight was recorded. The mixture was soaked for 4 hours, followed by sonication (power: 250 W, frequency: 40 kHz) for 20 minutes. The flask was removed, cooled, and weighed again. The lost weight was replenished with methanol, shaken well, and filtered. The subsequent filtrate was collected thereby obtaining the test solution.

Assay method: Precisely 1 μL each of the reference solution and the test solution was injected into the liquid chromatograph for determination, and the result was obtained accordingly.

### (4). Tetrahydropalmatine and Corydaline

The chromatographic conditions described under above section A (Chromatographic conditions and System Suitability Test) were used, with the column temperature maintained at 40° C. A triple quadrupole mass spectrometer equipped with an electrospray ionization source in positive mode (ESI⁺) was used. MRM was performed using the ion pairs m/z 356.1→159.0 and m/z 356.1→159.0. The theoretical plate number of the column was not less than 1000, calculated with reference to the tetrahydropalmatine peak.

The column temperature was maintained at 40° C. MRM was performed using the ion pairs m/z 370.2→170.0 and m/z 370.2→170.0. The theoretical plate number of the column was not less than 1000, calculated with reference to the corydaline peak.

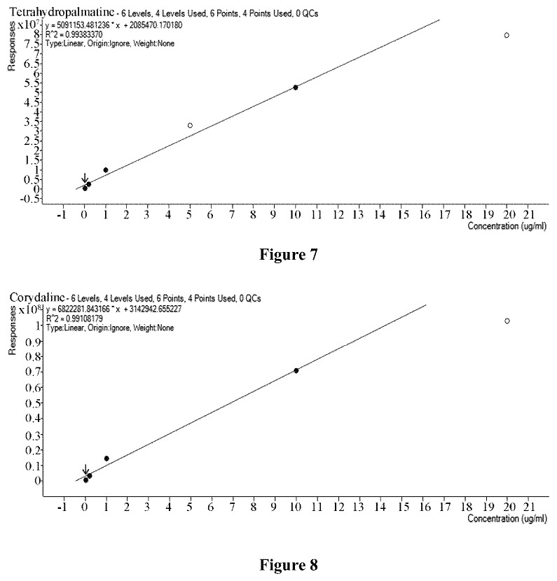

Preparation of reference solutions: appropriate amounts of tetrahydropalmatine and corydaline reference standards were accurately weighed and dissolved in methanol to prepare a series of standard solutions containing 6.0, 15.0, 30.0, 60.0, and 90.0 μg/mL of each compound, thereby obtaining the reference solutions. FIG. 7 shows the calibration curve of tetrahydropalmatine; FIG. 8 shows the calibration curve of corydaline.

Preparation of test solution: about 0.5 g of the PCME was accurately weighed and placed in a stoppered conical flask. Then, an accurate amount of 50 mL of a concentrated ammonia-methanol (1:20) mixture was added. The total weight was recorded. The mixture was cold-soaked for 1 hour, followed by reflux extraction in a water bath at 80° C. for 1 hour. The flask was removed, cooled, and weighed again. The lost weight was replenished with same ammonia-methanol mixture, shaken well, and filtered. The filtrate was evaporated to dryness in an evaporating dish. The residue was dissolved in methanol, transferred to a 5 mL volumetric flask, and diluted to volume with methanol. The solution was mixed well and filtered. The subsequent filtrate was collected thereby obtaining the test solution.

Assay method: Precisely 1 μL each of the reference solution and the test solution was injected into the liquid chromatograph for determination, and the result was obtained accordingly.

### EXAMPLE B: EVALUATION OF THE EFFECTS OF CDD-2105 FORMULATIONS

### 1. Materials

CDD-2105 solutions: 0.5% carboxymethylcellulose sodium (CMC-Na) solution was used to prepare various solutions of CDD-2105 formulations, including a CCMG formulation (Concentrated Chinese Medicine Granules, CCMG), a PCME formulation (Purified Chinese Medicine extracts, PCME), and a Q-marker formulation (Q-markers Mixture, MM). The volume of gavage administration for mice was 15 ml/kg.

Loperamide solution: the loperamide solution was prepared at a concentration of 0.5 mg/ml. The gavage administration volume for mice was 5 ml/kg.

Linaclotide solution: the linaclotide solution was prepared at a concentration of 3.97 μg/ml in 0.5% CMC-Na solution. The gavage administration volume for mice was 15 ml/kg.

### 2. Statistical Analysis

GraphPad 10 software was used for statistical analysis, and the results were expressed by mean±SEM. One-way ANOVA and Dunnett's multiple comparisons test were used for the analysis of differences between groups for stool frequency, fecal water content, and latency and Two-way ANOVA and Dunnett's multiple comparisons test were used for body weight and organ coefficient. A significance level of P<0.05 was considered statistically significant.

### Example B-1: Evaluation of the Anti-Constipation Effects of CDD-2105 Formulations

### (1) Treatment with CDD-2105 CCMG Formulations

C57BL/6J male mice aged 6˜8 weeks were randomly divided into 6 groups (n=8 per group) including: a control group, a loperamide group (Lop), Lop+CDD-2105 CCMG_1 group, Lop+CDD-2105 CCMG_2 group, Lop+CDD-2105 CCMG_3 group, and Lop+CDD-2105 CCMG_4 group. All CDD-2105 CCMG treated groups were given the CCMG by intragastric administration. The raw materials contained in the various CCMG formulations of CDD-2105 and the treatment of each group are as listed in below Tables 5-6. The Corydalis Rhizoma used in this example is Yanhusuo.

TABLE 5
Amount of raw materials in the CDD-2105 CCMG formulation

| | Amount of raw materials | | | |
|---|---|---|---|---|
| | Corydalis | Paeoniae | Magnoliae | Sennae |
| Formulation | Rhizoma | Radix Rubra | Officinalis Cortex | Folium |
| CDD-2105 | 5 g | 15 g | 10 g | 1 g |
| CCMG_1 | | | | |
| CDD-2105 | 15 g | 25 g | 20 g | 10 g |
| CCMG_2 | | | | |
| CDD-2105 | 10 g | 20 g | 15 g | 8 g |
| CCMG_3 | | | | |
| CDD-2105 | 10 g | 20 g | 15 g | 2 g |
| CCMG_4 | | | | |

TABLE 6
Administration protocol

| Group | Treatment 1 | Treatment 2 |
|---|---|---|
| Control | Mili-Q water, 5 ml/kg | 0.5% CMC-Na, 15 ml/kg |
| Loperamide (Lop) | loperamide, 5 ml/kg. | 0.5% CMC-Na, 15 ml/kg |
| Lop + CDD-2105 | loperamide, 5 ml/kg | CDD-2105 CCMG_1, |
| CCMG_1 | | 15 ml/kg |
| Lop + CDD-2105 | loperamide, 5 ml/kg | CDD-2105 CCMG_2, |
| CCMG_2 | | 15 ml/kg |
| Lop + CDD-2105 | loperamide, 5 ml/kg | CDD-2105 CCMG_3, |
| CCMG_3 | | 15 ml/kg |
| Lop + CDD-2105 | loperamide, 5 ml/kg | CDD-2105 CCMG_4, |
| CCMG_4 | | 15 ml/kg |

All drugs were administered by intragastric gavage (i.g.).

Treatments 1 and 2 were performed, with treatment 2 administered immediately after treatment 1.

### (2) Treatment with CDD-2105 PCME Formulations

C57BL/6J male mice aged 6˜8 weeks were randomly divided into 6 groups (n=10 per group) including: a control group, a loperamide group (Lop), Lop+CDD-2105 CCMG_4 group, Lop+CDD-2105 PCME (0.25×), Lop+CDD-2105 PCME (0.5×), and Lop+CDD-2105 PCME (1×). The content of the Q-Markers in each formulation and the administration of each group were listed in Tables 7-8.

TABLE 7
Concentrations of the Q-Markers in the formulation

| | THP | Magnolol | Sennoside | Paeoniflorin |
|---|---|---|---|---|
| Formulation | (mg/ml) | (mg/ml) | B (mg/ml) | (mg/ml) |
| CDD-2105 CCMG_4 | 0.0112 | 0.1436 | 0.1449 | 2.7389 |
| CDD-2105 | 0.0028 | 0.0359 | 0.0362 | 0.6847 |
| PCME (0.25×) | | | | |
| CDD-2105 | 0.0056 | 0.0718 | 0.0725 | 1.3695 |
| PCME (0.5×) | | | | |
| CDD-2105 | 0.0112 | 0.1436 | 0.1449 | 2.7389 |
| PCME (1×) | | | | |
| CDD-2105 | 0.0225 | 0.2872 | 0.2899 | 5.4778 |
| PCME (2×) | | | | |

TABLE 8
Administration protocol

| Group | Treatment 1 | Treatment 2 |
|---|---|---|
| Control | Mili-Q water, 5 ml/kg | 0.5% CMC-Na, 15 ml/kg |
| Loperamide (Lop) | loperamide, 5 ml/kg. | 0.5% CMC-Na, 15 ml/kg |
| Lop + CDD-2105 | loperamide, 5 ml/kg | CDD-2105 CCMG_4, |
| CCMG_4 | | 15 ml/kg |
| Lop + CDD-2105 | loperamide, 5 ml/kg | CDD-2105 PCME |
| PCME (0.25×) | | (0.25×), 15 ml/kg |
| Lop + CDD-2105 | loperamide, 5 ml/kg | CDD-2105 PCME |
| PCME (0.5×) | | (0.5×), 15 ml/kg |
| Lop + CDD-2105 | loperamide, 5 ml/kg | CDD-2105 PCME |
| PCME (1×) | | (1×), 15 ml/kg |

All drugs were administered by i.g.

Treatments 1 and 2 were performed, with treatment 2 administered immediately after treatment 1.

### (3). Stool Frequency

On the day of the experiments, the number of fecal pellets of mice within 6 h after administration of the formulations was recorded as stool frequency.

### (4). Water Content

On the day of the experiments, fecal samples of mice within 6 hours after administration of the formulations were collected in 2 ml of centrifuge tubes with a known weight, and the wet weight of the feces was recorded. After drying overnight at 105° C., the dry weight of the feces was recorded, and fecal water content was calculated to compare the fecal moisture of each group.

### (5). Results

### CDD-2105 Alleviates Loperamide-Induced Constipation in Mice

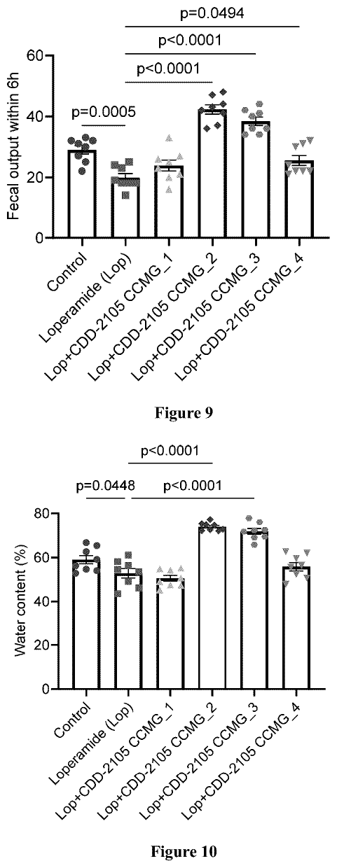

As shown in FIGS. 9-10, compared with the Lop group, CDD-2105 CCMG_2, _3, and _4 each significantly improved the stool frequency in loperamide-induced constipated mice. Moreover, CCMG_2 and _3 also significantly elevated the fecal water content.

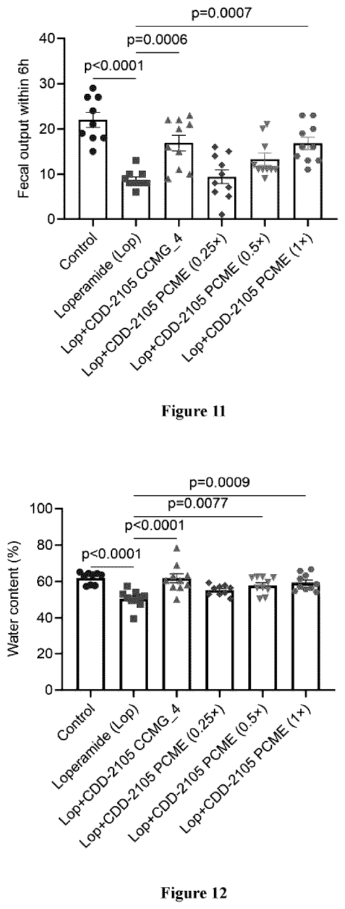

Due to the diarrhea of CDD-2105 CCMG_2 and _3 groups were more severe, in the subsequent experiments with CDD-2105 PCME formulations, CDD-2105 PCME (1×), whose Q-marker content was equivalent to CDD-2105 CCMG_4 was used. As seen from FIGS. 11-12, CDD-2105 PCME (1×) significantly increased the stool frequency of loperamide-induced constipated mice compared to the Lop group, and the effect was comparable to that of the CDD-2105 CCMG_4 (FIG. 11). Except for the CDD-2105 PCME (0.25×), other dosages of CDD-2105 PCME significantly increased the fecal water content of loperamide-constipated mice (FIG. 12)

These results suggested that CDD-2105 formulations were effective in alleviating constipation-like symptoms in mice.

### Example B-2: Evaluation of the Analgesic Effects of CDD-2105 Formulations in Mice

### (1) Treatment with CDD-2105 CCMG Formulations

C57BL/6J male mice aged 6-8 weeks with a basal pain threshold ranging from 5 s to 30 s were randomly divided into five groups (n=8 per group) including: a control group, CDD-2105 CCMG_1 group, CDD-2105 CCMG_2 group, CDD-2105 CCMG_3 group, and CDD-2105 CCMG_4 group. The content of raw materials in each of the CDD-2105 CCMG formulations was described in Table 5. The treatment of each group is as described in Table 9 below. All components or compositions (CMC-Na, and CDD-2105 CCMG) were administered by i.g.

TABLE 9
Administration protocol

| Group | Treatment |
|---|---|
| Control | 0.5% CMC-Na, 15 ml/kg |
| CDD-2105 CCMG_1 | CDD-2105 CCMG_1, 15 ml/kg |
| CDD-2105 CCMG_2 | CDD-2105 CCMG_2, 15 ml/kg |
| CDD-2105 CCMG_3 | CDD-2105 CCMG_3, 15 ml/kg |
| CDD-2105 CCMG_4 | CDD-2105 CCMG_4, 15 ml/kg |

### (2) Treatment with CDD-2105 PCME Formulations

C57BL/6J male mice aged 6˜8 weeks with basal pain threshold ranging from 5 s to 30 s were randomly divided into five groups (n=12 per group) including: a control group, a linaclotide group, CDD-2105 PCME (0.5×) group, CDD-2105 PCME (1×) group, and CDD-2105 PCME (2×) group. The content of the Q-Markers in each of the CDD-2105 PCME formulations is the same as that described in Table 7. The dosage of each group is as described in below Table 10. All components or compositions (CMC-Na, linaclotide, and CDD-2105 PCME) were administered by i.g.

TABLE 9
Administration protocol

| Group | Treatment |
|---|---|
| Control | 0.5% CMC-Na,15 ml/kg |
| Linaclotide | 3.97 μg/ml of linaclotide, 15 ml/kg |
| CDD-2105 PCME (0.5×) | CDD-2105 PCME (0.5×), 15 ml/kg |
| CDD-2105 PCME (1×) | CDD-2105 PCME (1×), 15 ml/kg |
| CDD-2105 PCME (2×) | CDD-2105 PCME (2×), 15 ml/kg |

### (3). Hot Plate Test

On the day of the experiment, mice were sequentially placed on a hot plate maintained at 55±1° C., 3 hours after gavage administration. The time to the first licking of the paws was recorded as the pain response latency (an indicator of pain threshold). If no licking behavior occurred within 30 s, the mouse was immediately removed from the hot plate to prevent burns, and the latency was recorded as 30 s.

### (4). Results

### CDD-2105 Alleviates Thermal-Stimulation-Induced Central Nociception in Mice

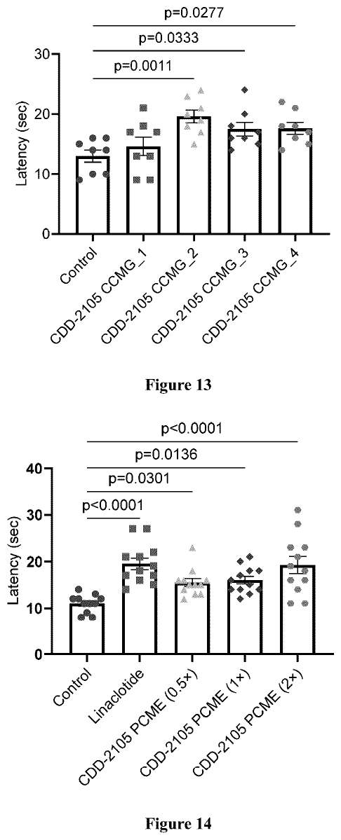

As shown in FIG. 13, the pain threshold of mice in CDD-2105 CCMG_2, _3, and _4 groups were significantly higher than that of the control group at 3 h after administration.

As shown in FIG. 14, the analgesic effects of CDD-2105 PCME formulations were shown in a dose-dependent manner at 3 h after administration, wherein the content of Q-markers in CDD-2105 PCME (1×) was equivalent to CDD-2105 CCMG_4. The effects of CDD-2105 PCME (2×) were comparable to that of the linaclotide.

These results indicated that CDD-2105 formulations exhibit a significant analgesic effect on central nociception in mice.

### Example B-3: Evaluation of Toxicity of CDD-2105 Q-Marker Formulations in Mice

### (1). Animals and Administration

ICR mice aged 6˜8 weeks (female: male=1:1) were randomly divided into five groups (n=4 per group) including: a control group, CDD-2105 Q-marker mixture (MM) (10×) group, CDD-2105 MM (20×) group, CDD-2105 MM (50×) group, and CDD-2105 MM (100×) group. The single dose was set according to the up-and-down procedure and listed in Table 11 below.

One day before administration, each of mice was weighed and the data was recorded. The death of animals, the changes in body weight, and food intake of mice in each group after administration were recorded. On the 14th day after oral administration, the heart, liver, spleen, lung, and kidney of mice were visually observed by the unaided eye and weighed. The organ coefficients were calculated.

The Q-markers including tetrahydropalmatine, magnolol, sennoside A, and paeoniflorin, were selected and mixed to form the Q-marker formulations of CDD-2105. The single dose of each formulation is shown in below Table 11.

TABLE 11
Doses of the formulation

| | The total content of Q- | Clinical |
|---|---|---|
| Formulation | markers (mg/kg) | equivalent dose |
| CDD-2105 MM (10x) | 924.33 | 10x |
| CDD-2105 MM (20x) | 1848.46 | 20x |
| CDD-2105 MM (50x), | 4621.15 | 50x |
| CDD-2105 MM (100x) | 9242.29 | 100x |

### (2). Results

### CDD-2105 Does not Have a Serious Acute Toxicity Risk

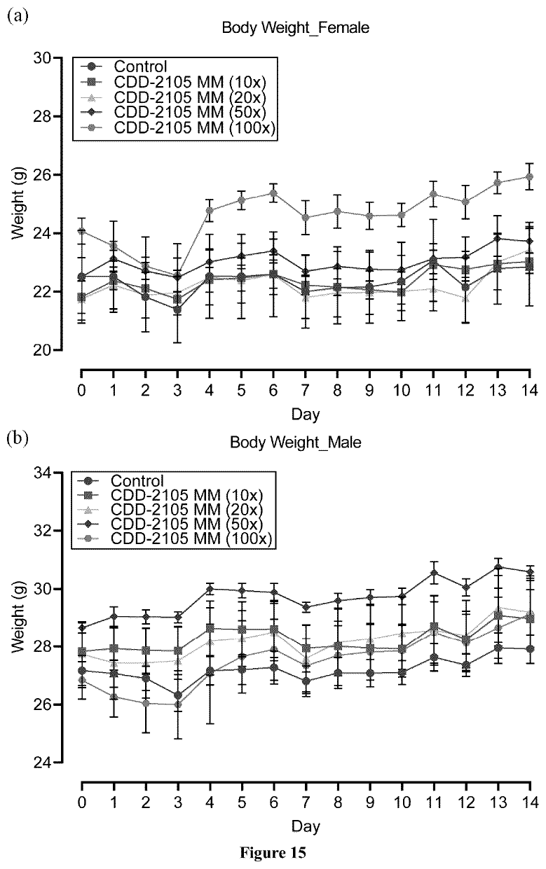

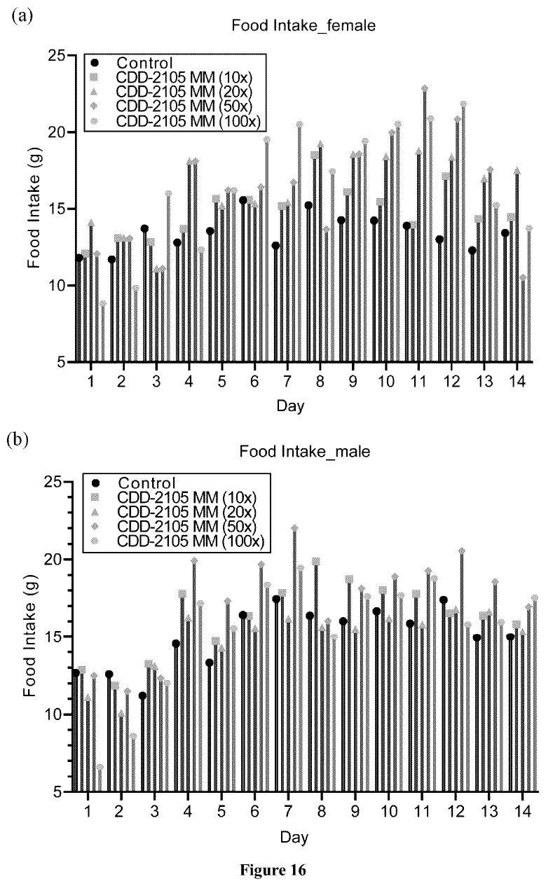

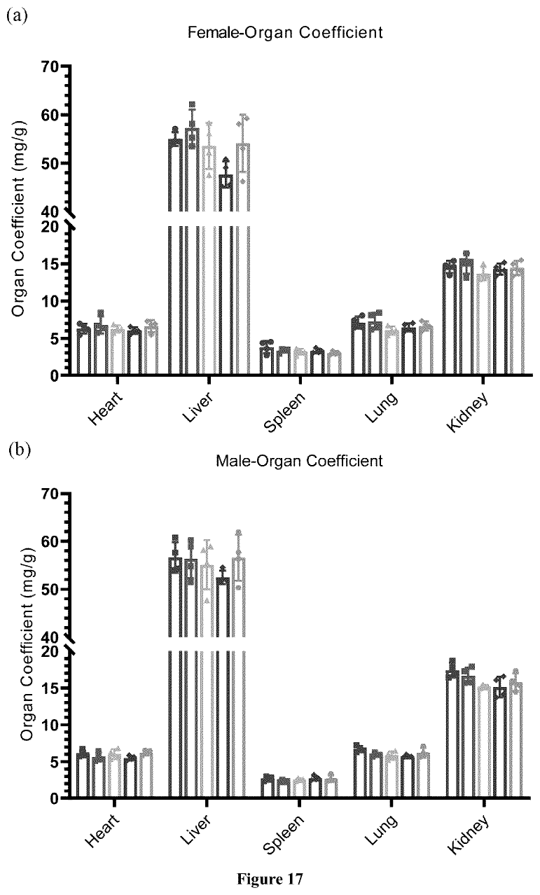

After a single administration of the Q-markers formulations of CDD-2105, no death of female and male mice was observed during the observation period. The body weight (FIG. 15) and food intake (FIG. 16) were not negatively affected. After 14 days of the administration, no obvious lesions were observed in the organs upon visual inspection, and organ coefficients were not obviously affected by CDD-2105 Q-Marker formulations (FIG. 17). The results indicate that CDD-2105 formulations have no risk of severe acute toxicity and exhibit a good safety profile.

What is claimed is:

1. A traditional Chinese medicine composition comprising by dry weight:
1 to 25 parts of Corydalis Rhizoma;
5 to 50 parts of Paeoniae Radix;
1 to 30 parts of Magnoliae Officinalis Cortex; and
1 to 20 parts of Sennae Folium,
wherein the Paeoniae Radix is Paeoniae Radix Rubra, Paeoniae Radix Alba, or a combination thereof, and
wherein the Corydalis Rhizoma is Yanhusuo, Cuyanhusuo, or a combination thereof.

2. The traditional Chinese medicine composition of claim 1 comprising by dry weight:
5 to 15 parts of Corydalis Rhizoma;
15 to 25 parts of Paeoniae Radix;
10 to 20 parts of Magnoliae Officinalis Cortex; and
1 to 10 parts of Sennae Folium.

3. The traditional Chinese medicine composition of claim 1 comprising by dry weight:
5 to 10 parts of Corydalis Rhizoma;
15 to 20 parts of Paeoniae Radix;
10 to 15 parts of Magnoliae Officinalis Cortex; and
1 to 2 parts of Sennae Folium.

4. The traditional Chinese medicine composition of claim 1 comprising by dry weight:
5 or 10 parts of Corydalis Rhizoma;
15 or 20 parts of Paeoniae Radix;
10 or 15 parts of Magnoliae Officinalis Cortex; and
1 or 2 parts of Sennae Folium.

5. The traditional Chinese medicine composition of claim 1 comprising:
0.1449 mg/mL sennoside B,
0.1436 mg/mL magnolol,
2.7389 mg/mL paeoniflorin, and
0.0112 mg/mL tetrahydropalmatine.

6. The traditional Chinese medicine composition of claim 1, wherein the composition is in a form selected from the group consisting of a solid mixture, a capsule, a tablet, a powder, a granule, and a liquid formulation.

7. A composition comprising by weight:
1-10% sennoside A,
1-10% sennoside B,
1-10% magnolol,
1-10% honokiol,
30-50% paeoniflorin,
5-10% albiflorin,
0.1-0.5% corydaline, and
0.1-0.5% tetrahydropalmatine.

8. The composition of claim 7, wherein the composition comprises by weight:
1-5% sennoside A,
1-5% sennoside B,
1-5% magnolol,
1-5% honokiol,
30-50% paeoniflorin,
5-10% albiflorin,
0.1-0.3% corydaline, and
0.1-0.3% tetrahydropalmatine.

9. The composition of claim 7, wherein the composition comprises by weight:
1-2% sennoside A,
1-3% sennoside B,
1-3% magnolol,
1-2% honokiol,
40-45% paeoniflorin,
5-7% albiflorin,
0.1-0.2% corydaline, and
0.1-0.2% tetrahydropalmatine.

10. The composition of claim 7, wherein the composition is obtained from a traditional Chinese medicine composition comprising: Corydalis Rhizoma; Paeoniae Radix; Magnoliae Officinalis Cortex; and Sennae Folium, wherein the Paeoniae Radix is Paeoniae Radix Rubra, Paeoniae Radix Alba, or a combination thereof, and wherein the Corydalis Rhizoma is Yanhusuo, Cuyanhusuo, or a combination thereof.

11. The composition of claim 7 further comprising a solvent selected from water and 0.5% carboxymethylcellulose sodium (CMC-Na) aqueous solution.

12. The composition of claim 11, wherein the composition comprises:
0.0725-0.2899 mg/mL sennoside B,
0.0718-0.2872 mg/mL magnolol,
1.3695-5.4778 mg/mL paeoniflorin, and
0.0056-0.0225 mg/mL tetrahydropalmatine.

13. A method of treating constipation in a subject in need thereof, the method comprising: administering a therapeutically effective amount of the composition of claim 1 to the subject.

14. The method of claim 13, wherein the constipation is irritable bowel syndrome with constipation (IBS-C), slow transit constipation, outlet obstruction constipation, mixed-type constipation, drug-induced constipation, secondary constipation, or functional constipation; or wherein the constipation is accompanied by abdominal pain.

15. A method of treating constipation in a subject in need thereof, the method comprising: administering a therapeutically effective amount of the composition of claim 7 to the subject.

16. The method of claim 15, wherein the constipation is irritable bowel syndrome with constipation (IBS-C), slow transit constipation, outlet obstruction constipation, mixed-type constipation, drug-induced constipation, secondary constipation, or functional constipation; or wherein the constipation is accompanied by abdominal pain.

17. A method of preparing the composition of claim 7, the method comprising:
combining Corydalis Rhizoma; Paeoniae Radix; Magnoliae Officinalis Cortex; and Sennae Folium, in a mass ratio of (5-15):(15-25):(10-20):(1:10) respectively to form a mixture, wherein the Paeoniae Radix is Paeoniae Radix Rubra, Paeoniae Radix Alba, or a combination thereof, and wherein the Corydalis Rhizoma is Yanhusuo, Cuyanhusuo, or a combination thereof; and
extracting sennoside A, sennoside B, magnolol, honokiol, paeoniflorin, albiflorin, corydaline, and tetrahydropalmatine from the raw materials to obtain the composition.
